(19) United States
(12) Patent Application Publication
(10) Pub. No.: US 2014/0057267 A1
(43) Pub. Date: Feb. 27, 2014

(54) COMPOSITION AND KIT FOR THE DIAGNOSIS OF MILD COGNITIVE IMPAIRMENT, WHICH MEASURE AN EXPRESSION LEVEL OF LIPOCALIN-2, AND METHOD FOR PROVIDING INFORMATION FOR THE DIAGNOSIS OF MILD COGNITIVE IMPAIRMENT

(71) Applicants: Kyoungho Suk, Suseong-gu Daegu (KR); Ho Won Lee, Beomeo-dong Suseong-gu Daegu (KR); Ji Hye Choi, Dong-gu Daegu (KR)

(72) Inventors: Kyoungho Suk, Suseong-gu Daegu (KR); Ho Won Lee, Beomeo-dong Suseong-gu Daegu (KR); Ji Hye Choi, Dong-gu Daegu (KR)

(73) Assignee: KYUNGPOOK NATIONAL UNIVERSITY INDUSTRY-ACADEMIC COOPERATION FOUNDATION, Daegu (KR)

(21) Appl. No.: 14/112,836

(22) PCT Filed: Apr. 2, 2012
(86) PCT No.: PCT/KR2012/002454

§ 371 Date: Nov. 8, 2013

(30) Foreign Application Priority Data
Apr. 22, 2011 (KR) 10-2011-0037775

(51) Int. Cl. G01N 33/68

(52) U.S. Cl. CPC G01N 33/6896
USPC 435/6.11; 435/6.12; 435/7.94

(57) ABSTRACT

The present invention relates to a composition for the diagnosis of mild cognitive impairment, which includes a formulation measuring an mRNA or protein expression level of lipocalin-2 gene, to a kit for the diagnosis of mild cognitive impairment, and to a method for providing information for the diagnosis of mild cognitive impairment using the same. According to the present invention, by using the agent for measuring an mRNA or protein expression level of the lipocalin-2 gene, a patient having mild cognitive impairment can be specifically identified by measuring an expression level of lipocalin-2, which is higher in a group of patients having mild cognitive impairment than in both a normal group and a group of patients having Alzheimer's disease.; In particular, it is possible to distinguish between a group of patients having mild cognitive impairment and a group of patients having Alzheimer's disease

### TECHNICAL FIELD

The present invention relates to a composition for diagnosing mild cognitive impairment, comprising an agent for measuring the level of mRNA or protein expression of lipocalin-2 gene, to a kit comprising the same, and to a method for providing information for diagnosing mild cognitive impairment, characterized by measuring the level of lipocalin-2 protein expression.

### BACKGROUND ART

Cognitive impairments in brain are characterized clinically by progressive loss of memory, cognition, reasoning, executive functioning, planning, judgment and emotional stability, gradually leading to profound mental deterioration. A wide range of disorders may lead to the cognitive impairment.

Neuropsychological cognitive deficits are common in people with functional neuropsychiatric disorders. Among these, schizophrenia is a chronic, severe and disabling form of psychosis. Scientists have estimated that up to 75% of schizophrenic patients are cognitively impaired. It is known that traditional treatments for schizophrenia are not effective to treat cognitive deficits in schizophrenia. While it has been reported that more recently developed treatments for schizophrenia, known as “atypical anti-psychotics”, may have some effect on cognitive deficits, the effect may not be lasting or not lead to an improvement in daily functioning. There are currently no drugs approved for the treatment of cognitive deficits in schizophrenia.

More in general across several pathological conditions, with the increase of medical screening for dementia, an increasing number of patients are being identified who do not meet the diagnostic criteria for dementia but nonetheless have significant memory or cognitive impairment, defined as mild cognitive impairment.

Mild cognitive impairment (MCI) is a condition characterized by mild recent memory loss without dementia or significant impairment of other cognitive functions to an extent that is beyond that expected for age or educational background. Criteria for diagnosis of MCI are memory complaint, abnormal activities of daily life, abnormal general cognitive functioning, abnormal memory for age, not demented, etc.

The number of patients falling in the categories of mild cognitive impairment, age-associated memory impairment, age-related cognitive decline or similar diagnostic categories is staggering. For example, according to the estimates of Barker et al. (Br J Psychiatry, 1995 November; 167(5):642-8), there are more than 16 million people with age-associated memory impairment in the U.S.

Lipocalin-2 is a member of the lipocalin family and is known to bind or transport lipid and other hydrophobic molecules (Flower et al., Biochem Biophys Acta 1482:9-24, 2000; Kjeldsen et al., Biochem Biophys Acta 1482:272-283, 2000). Moreover, lipocalin-2 is also known as 24p3 (Flower et al., Biochem Biophys Res Commun 180:69-74, 1991), 24 kDa superinducible protein (SIP24) (Hamilton et al., J Cell Physiol 123:201-208, 1985), and neutrophil gelatinase-associated lipocalin (NGAL; a human homologue of 1cn2) (Kjeldsen et al., J Biol Chem 268:10425-10432, 1993; Borregaard and Cowland, Biometals 19:211-215, 2006).

It has been reported that lipocalin-2 has diverse functions and thus is important for both cellular apoptosis and survival (Devireddy et al., Science 293:829-834, 2001; Yousefi and Simon, Cell Death Differ 9:595-597, 2002; Tong et al., Biochem J 372:203-210, 2003; Devireddy et al., Cell 123:1293-1305, 2005; Tong et al., Biochem J 391:441-448, 2005). Moreover, it has been reported that lipocalin-2 also plays a central role in the inducing cellular differentiation in the kidney during embryogenesis (Yang et al., Mol Cell 10:1045-1056, 2002) and protects the kidney from ischemic injury (Mishra et al., J Am Soc Nephrol 15:3073-3082, 2004; Mori et al., J Clin Invest 115:610-621, 2005). In various forms of gastrointestinal injury, lipocalin-2 facilitates mucosal regeneration by promoting cell migration (Playford et al., Gastroenterology 131:809-817, 2006). However, no correlation has been reported between the lipocalin-2 and mild cognitive impairment.

An advisory panel to the US Food and Drug Administration ruled on Tuesday, Mar. 13, 2001 that mild cognitive impairment, “a condition separate from dementia in Alzheimer's disease (AD)”, is a valid target for new drug therapies, regardless of whether a particular drug also slows the progression to dementia. However, no specific diagnosis of mild cognitive impairment, which is distinguished from dementia, and no method for distinguishing mild cognitive impairment from Alzheimer's disease have been reported.

As such, if Alzheimer's disease can be distinguished from mild cognitive impairment, which is a prodromal stage of Alzheimer's disease, it is possible to specifically diagnose and effectively treat mild cognitive impairment, and thus it is necessary to develop a new target for such diagnosis.

### DISCLOSURE

### Technical Problem

The present inventors have studied a target for specifically diagnosing mild cognitive impairment, which is distinguished from Alzheimer's disease, and found that the measurement of the level of lipocalin-2 expression allows to specifically diagnose mild cognitive impairment and to distinguish mild cognitive impairment from Alzheimer's disease, thus completing the present invention.

Therefore, an object of the present invention is to provide a composition for diagnosing mild cognitive impairment, characterized by measuring the level of lipocalin-2 expression, a kit for comprising the same, and a method for providing information for diagnosing mild cognitive impairment.

### Technical Solution

The present invention provides a composition for diagnosing mild cognitive impairment, comprising an agent for measuring the level of mRNA or protein expression of lipocalin-2 gene and a kit comprising the same.

Moreover, the present invention provides a method for providing information for diagnosing mild cognitive impairment, characterized by measuring the level of lipocalin-2 protein.

### Advantageous Effects

The composition for diagnosing mild cognitive impairment, comprising an agent for measuring the level of mRNA or protein expression of lipocalin-2 gene according to the present invention and the kit comprising the same can specifically distinguish a patient with mild cognitive impairment, and in particular can distinguish patients with Alzheimer's disease from patients with mild cognitive impairment by measuring the level of lipocalin-2 expression, which exhibits a higher level of expression in patients with mild cognitive impairment compared to normal subjects and patients with Alzheimer's disease.

### DESCRIPTION OF DRAWINGS

FIG. 1 is a diagram showing the results of plasma lipocalin-2 levels by sandwich ELISA in the control group, in the mild cognitive impairment patient group, and in the Alzheimer's disease patient group.

FIG. 2 is a diagram showing the comparison of plasma lipocalin-2 levels in mild Alzheimer's disease patients and in severe Alzheimer's disease patients.

FIG. 3 is a diagram showing the correlation between lipocalin-2 levels and mini-mental state examination (MMSE) scores.

FIG. 4 is a diagram showing the correlation between plasma lipocalin-2 levels and clinical dementia rating (CDR) scores.

FIG. 5 is a diagram showing the correlation between plasma lipocalin-2 levels and ages in the control group.

FIG. 6 is a diagram showing the correlation between plasma lipocalin-2 levels and cerebrospinal fluid lipocalin-2 levels.

### DETAILED DESCRIPTION OF THE INVENTION

The present invention provides a composition for diagnosing mild cognitive impairment, comprising an agent for measuring the level of mRNA or protein of lipocalin-2 gene.

“Mild cognitive impairment (MCI)” of the present invention is a disease that is not necessarily related to the presence of dementia, characterized by mild but measurable impairment of cognitive functioning. Mild cognitive impairment may frequently, but not necessarily, frequently lead to Alzheimer's disease.

“Mini—mental state examination (MMSE)” of the present invention is one of the simple dementia screening tests and is a test method that is applicable to patients with severely impaired functions and provides quantified information on overall functional levels of patients. Especially, it refers to a cognitive screening test. In general, an MMSE score of greater than 25 indicates a normal cognitive status, and an MMSE score of less than 12 indicates sever Alzheimer's disease.

“Clinical dementia rating (CDR) scores” of the present invention refer to a clinical dementia rating scale. This scale is characterized in that it is based on clinical information, but pathological findings, etc. are excluded. It is a method for measuring cognitive performance, and a higher score indicates a more severe degree of dementia.

“One-way analysis of variance (ANOVA)” of the invention refers to an analysis method that is used when there are one independent variable and two or more populations of independent variables.

“Comorbidity” may be assessed by the Charlson Index of Comorbidity referred to as the Comorbidity Index, and assessable items may include myocardial infarction, heart failure, vascular disease, hypertension, chronic obstructive pulmonary disease, arthritis, gastrointestinal disease, mild liver disease, diabetes, chronic renal disease, and systemic malignancy.

The level of lipocalin-2 expression of the present invention may be measured by immunological analysis, hybridization, and amplification at the protein and/or mRNA level and may be measured by various analytical methods known in the art without limitation. Preferably, the detection of lipocalin-2 nucleic acid may be performed by amplification using one or more oligonucleotide primers which hybridize to nucleic acid molecules encoding lipocalin-2 or complements thereof.

More specifically, the detection of lipocalin-2 nucleic acid using primers may be performed by amplifying lipocalin-2 gene sequences using PCR amplification and determining whether the genes are amplified by a method known in the art.

Therefore, the present invention provides a composition for diagnosing mild cognitive impairment, comprising a pair of primers or a probe that is specific to the lipocalin-2 gene.

As used herein, the term “primer” refers to a short nucleic acid sequence having a free hydroxyl group, which is able to undergo base-pairing interaction with a complementary template and serves as a starting point for replicating the template strand. The primer for amplifying lipocalin-2 nucleic acid may be easily prepared by a method known in the art. A suitable primer can amplify a portion of the nucleic acid molecule and is based on a nucleic acid sequence encoding at least 7 consecutive amino acids of lipocalin-2 nucleic acids. In general, the primer has a length of 17-25 bp, preferably 20 bp, and has a sequence homology of about 60%, preferably more than 75%, more preferably more than 90% to polynucleotide encoding lipocalin-2.

Examples of the method for detecting lipocalin-2 genes using the primer may include, but not limited to, polymerase chain reaction (PCR), DNA sequencing, RT-PCR, primer extension (Nikiforeov et al., Nucl Acids Res 22, 4167-4175, 1994), oligonucleotide extension analysis (Nickerson et al., Pro Nat Acad Sci USA, 87, 8923-8927,1990), allele-specific PCR (Rust et al., Nucl Acids Res, 6, 3623-3629, 1993), RNase mismatch cleavage (RNase mismatch cleavage, Myers et al., Science, 230, 1242-1246, 1985), single strand conformation polymorphism (SSCP, Orita et al., Pro Nat Acad Sci USA, 86, 2766-2770, 1989) and heteroduplex simultaneous analysis (Lee et al., Mol Cells, 5:668-672, 1995), denaturing gradient gel electrophoresis (DGGE, Cariello et al., Am J Hum Genet, 42, 726-734, 1988), denaturing high pressure liquid chromatography (Underhill et al., Genome Res, 7, 996-1005, 1997), hybridization reaction, DNA chip, etc. Examples of the hybridization reaction may include northern hybridization (Maniatis T. et al., Molecular Cloning, Cold Spring Habor Laboratory, NY, 1982), in situ hybridization (Jacquemier et al., Bull Cancer, 90:31-8, 2003), microarray (Macgregor, Expert Rev Mol Diagn 3:185-200, 2003), etc. The PCR may include deoxynucleotide triphosphate (dNTP), thermostable polymerase, salts of metal ions such as magnesium chloride, etc., which are required for the PCR reaction, and include dNTP, sequenase, which are required for the sequencing.

Moreover, the diagnostic composition of the present invention may be immobilized on a suitable carrier or support in order to enhance the rapidness and convenience of diagnosis (Antibodies: A Laboratory Manual, Harlow & Lane; Cold SpringHarbor, 1988). Examples of suitable carriers or supports may include agarose, cellulose, nitrocellulose, dextran, Sephadex, Sepharose, liposomes, carboxymethyl cellulose, polyacrylamides, polystyrene, gabbros, filter paper, ion-exchange resin, plastic film, plastic tube, glass, polyamine-methyl vinyl-ether-maleic acid copolymer, amino acid copolymer, ethylene-maleic acid copolymer, nylon, cups, flat packs, etc. Other solid substrates may include cell culture plates, ELISA plates, tubes, and polymeric membranes. The support may have any possible form such as spherical (e.g., bead), cylindrical (e.g., inside surface of a test tube or well, or flat (e.g., sheet, test strip).

Preferably, the composition for diagnosing mild cognitive impairment, comprising an antibody specific to lipocalin-2 protein may be provided in the form of a kit.

The diagnostic kit may be provided in the form of a lateral flow assay kit based on immunochromatography to detect a lipocalin-2 protein in a plasma sample. The lateral flow assay kit may comprise a sample pad to which the plasma sample is applied, a releasing pad which is coated with an antibody for detection, a developing membrane (e.g., nitrocellulose) or strip in which the sample is transferred and separated and an antigen-antibody reaction occurs, and an absorption pad.

Moreover, the present invention provides a method for providing information for diagnosing mild cognitive impairment, characterized by measuring the level of lipocalin-2 protein. Preferably, the information providing method may comprise the steps of measuring the level of lipocalin-2 protein in a biological sample; and determining an increase in lipocalin-2 protein by comparison with a normal control group. More preferably, the biological sample may be plasma or cerebrospinal fluid for the diagnosis of mild cognitive impairment.

To measure the level of lipocalin-2 protein expression according to the present invention, an antibody specific to lipocalin-2 protein may preferable be used. The antibody of the present invention may include both a monoclonal antibody and a polyclonal antibody.

### Mode for Invention

Hereinafter, the present invention will be described in detail with reference to Examples. However, the following Examples are to illustrate the present invention, and the present invention is not limited by the following Examples.

### EXAMPLE 1

### Recruitment of Experimental Subjects and Experimental Methods

1-1. Recruitment of Patients for Screening

Participants for the screening of the present invention were recruited from patients who visited the Dementia Clinic of Kyungpook National University Hospital. The screening was performed on a total of 141 patients, and the classification of the patients is shown in the following table 1:

TABLE 1

| mild cognitive | | |
|---|---|---|
| impairment patients | Alzheimer's disease patients | Normal subjects |
| 41 | 62 | 38 |

These participants were evaluated and classified into the above groups by neuropsychological evaluation, psychiatric evaluation and interview, blood analyses including apolipoprotein E, brain magnetic resonance images, etc. Disease comorbidity was assessed by the Charlson Index of Comorbidity referred to as the Comorbidity Index, and the neuropsychological evaluation was performed using the clinical dementia rating (CDR) and the mini-mental state examination (MMSE). The characteristics of the participants are shown in the following table 2:

TABLE 2

| | Controls (n = 38) | MCI (n = 41) | AD (n = 62) |
|---|---|---|---|
| Gender (M/F) | 15/23 | 18/23 | 16/46 |
| Age (years) | 64.92 ± 5.57 | 69.02 ± 7.77 | 72.16 ± 6.35 |
| MMSE score | 28.45 ± 1.52 | 24.80 ± 3.28 | 15.69 ± 4.43 |
| CDR score | 0.16 ± 0.23 | 0.5 ± 0.07 | 1.33 ± 0.57 |
| Education (years) | 10.85 ± 3.46 | 8.87 ± 5.32 | 4.24 ± 3.70 |

Abbreviations:
MMSE, mini-mental state examination;
CDR, clinical dementia rate;
BMI, body mass index;
MCI, mild cognitive impairment;
AD, Alzheimer's disease.
Values are mean ± S.D.

1-2. Collection of Plasma Samples

Plasma samples from patients fasting for more that 8 hour were collected into sodium heparin tubes early in the morning and separated by centrifuging the samples at 2,000 rpm for 15 minutes. Thereafter, the supernatants were separated and stored at −80° C. until use in the experiments. Cerebrospinal fluid (CSF) samples were collected through a lumbar puncture. Plasma and CSF samples were stored at −80° C. pending biochemical analysis, without being thawed and re-frozen.

1-3. Measurement of Lipocalin-2 in Cerebrospinal Fluid and Plasma

The lipocalin-2 levels in plasma (1:400 dilution) and cerebrospinal fluid (1:2 dilution) were measured using a Sandwich ELISA Duo-set (purchased from R&D systems; Minneapolis, Minn.). Primary antibodies (rat anti-human lipocain-2) diluted in PBS at room temperature overnight, plated in 96-well Elisa plates, and washed three times with PBS-T (phosphate buffered saline with 0.05% Tween 20). Blocking was performed with PBS containing 1% bovine serum albumin (BSA) at room temperature for 1 hours, followed by washing with PBS-T three times. For standards, human recombinant lipocalin-2 was used at concentrations ranging from 39.06 to 2500 pg/ml. 100 μl of the sample was placed in each well and washed three times with PBS-T after reaction at room temperature for 2 hours. Then, 100 μl of secondary antibody (biotinylated goat anti-human IgG) was added to each well and washed three times with PBS-T after reaction at room temperature for 2 hours. Thereafter, horseradish peroxidase-conjugated streptavidin was added, washed three times with PBS-T after reaction for 20 minutes, and then washed three times with PBS-T. Lastly, 100 μl of mixture of 3,3′,5,5′-tetramethylbenzidine (TMB) as a peroxidase substrate and H₂O₂ as a peroxidase solution in a ratio of 1:1 was added, and the reaction was stopped by adding 2N H₂SO₄, and the absorbance was measured at a wavelength of 450 nm. All experiments were analyzed using mean values from duplicate measurements, the protein concentration in each patient was measured by Bradford assay, and the lipocalin-2 levels were adjusted for the protein concentration in each patient and used for comparison and analysis.

The comparison for lipocalin-2 protein levels between the control group, the mild cognitive impairment group, and the Alzheimer's disease group was done by one-way analysis of variance (ANOVA) with Turkey-HSD test for post hoc comparisons. In addition to the variable group, clinical data was added as a predictor, and age, gender, BMI, years of education, and comorbidity were added as a covariate in the analysis of the covariance models. The Spearman's analyses for correlations were also done on a subset of participants with all available data on the relationship between LCN2 levels, MMSE, and CDR score using linear regression for the covariates.

Statistical analyses were done using SPSS 17.0 software (SPSS Inc; Chicago, Ill.), Sigma plot 10.0 (SPSS Inc; Chicago, Ill.), and MATLAB 7.0 (The Mathworks; Natick, Mass.). The statistical significance value (p) was set at <0.05. Results were expressed as the mean±SD.

### EXAMPLE 2

### Diagnosis of Mild Cognitive Impairment by Analysis of Lipocalin-2 Expression

2-1. Comparison of Levels of Lipocalin-2 Protein Expression

The difference in the level of lipocalin-2 protein expression between three groups (mild cognitive impairment group, Alzheimer's group, and control group) compared by one-way analysis of variance (ANOVA) and represented in plasma lipocalin-2 levels (p=0.001). As a result, there were no significant differences in gender and body mass index (BMI; kg/m2) between the three groups, but there were significant differences in age, years of education, and comorbidity between the three groups (p<0.0001; p<0.0001; and p<0.0001, respectively).

Analyses were adjusted for age and comorbidity.

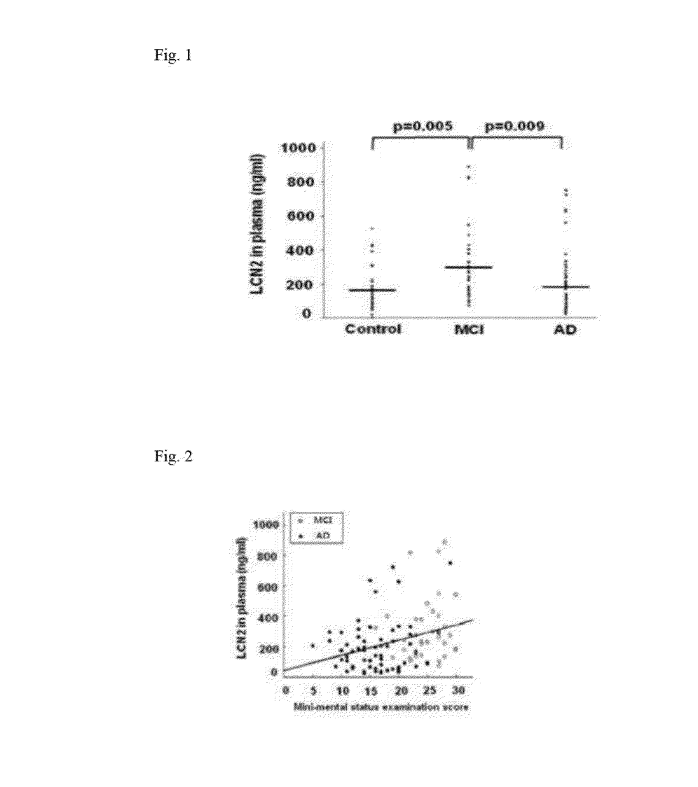

The results are shown in FIG. 1.

As shown in FIG. 1, the levels of lipocalin-2 expression in plasma had statistically significant differences between the three groups (the control group: n=38, 163 ng/ml; the mild cognitive impairment group: n=41, 296 ng/ml; and the Alzheimer's group: n=62, 191 ng/ml (p=0.005, p=0.009, respectively).

Moreover, the MMSE scores and plasma lipocalin-2 levels in accordance with the degree of the cognitive impairment that indicates the severity of Alzheimer's disease symptoms were compared.

The results are shown in FIG. 2.

As shown in FIG. 2, in the case of MMSE≧12, classified as mild Alzheimer's disease, the plasma lipocalin-2 level was 195.58 ng/ml, and in the case of MMSE<12, classified as severe Alzheimer's disease, the plasma lipocalin-2 level was 170.58 ng/ml (p=0.049 based on Student's t-test).

2-2. Analysis of Correlation Between Plasma Lipocalin-2, MMSE, and CDR

Correlation was evaluated to assess the associations between the clinical data (MMSE and CDR) and the plasma lipocalin-2 levels.

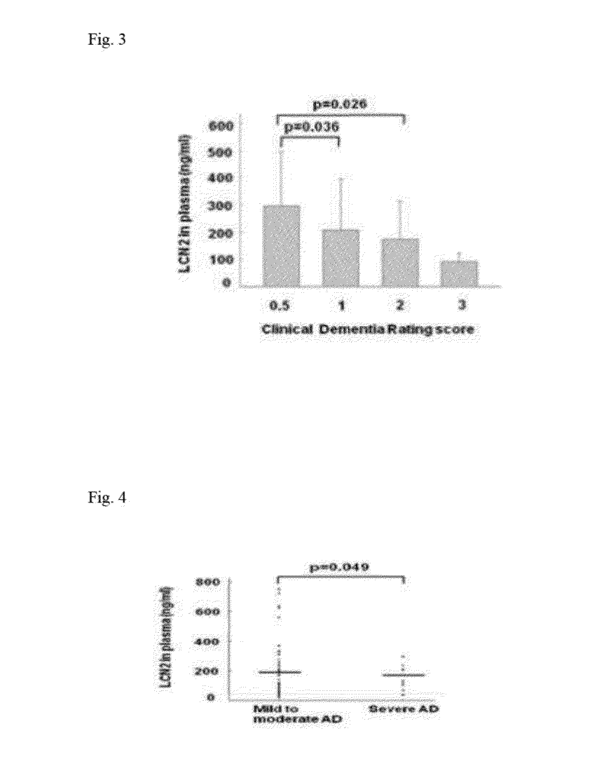

The results are shown in FIGS. 3 and 4.

As shown in FIG. 3, In patients with Alzheimer's disease or mild cognitive impairment, no correlation was found between the lipocalin-2 plasma concentration and MMSE score (Spearman's correlation rho=0.017 and p=0.893 for AD; rho=0.247 and p=0.124 for MCI, respectively). Moreover, there were no significant correlations between the MMSE scores and lipocalin-2 levels in any of the three groups such as the Alzheimer's disease group, the mild cognitive impairment group, and the control group, separately. However, there were significant positive correlations between the MMSE scores and lipocalin-2 levels in the two groups together; the Alzheimer's disease group and the mild cognitive impairment group (rho=0.317, p=0.001).

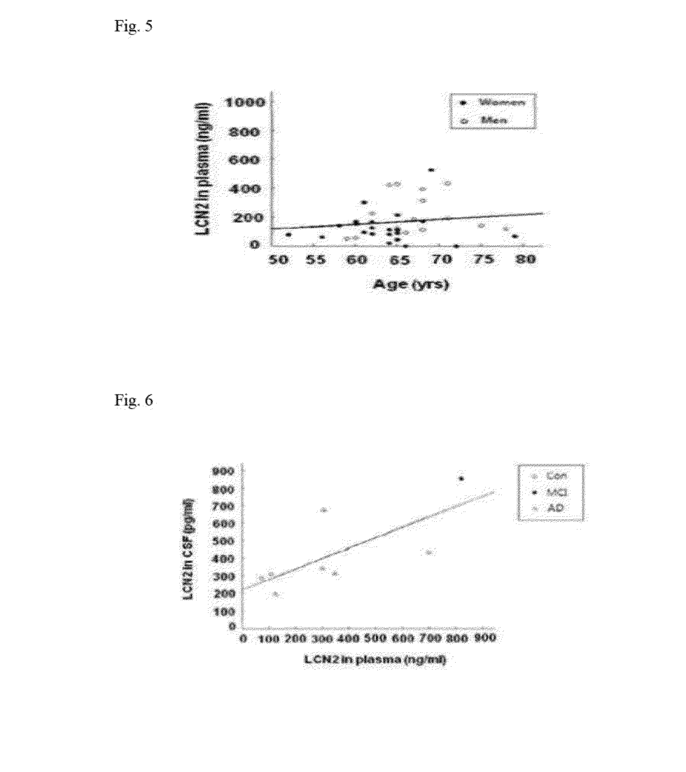

Moreover, as shown in FIG. 4, there was a significant negative correlation between the plasma lipocalin-2 levels and the CDR scores (Spearman's correlation rho=0.245, p=0.014). On the contrary, the severity of Alzheimer's disease correlates negatively with MMSE score, and positively with CDR scores. Based on this, it was found that the plasma lipocalin-2 exhibited a higher level of expression in the mild cognitive impairment group, compared to the Alzheimer's disease group and the control group. Moreover, and the plasma level exhibited a higher level of expression in the mild Alzheimer's disease group, compared to the severe Alzheimer's disease group. There were differences in plasma lipocalin-2 levels between the three groups such as the Alzheimer's disease group, the mild cognitive impairment group, and the control group, and both the Alzheimer's disease group, the mild cognitive impairment group exhibited higher plasma lipocalin-2 levels compared to the control group. It is known that the incidence rate of Alzheimer's disease increases with age, inflammatory reaction occurs in increased senile plaques and neurofibrillary tangles, which result in the release of inflammatory stimuli, and these inflammatory stimuli are increased in the blood. However, as shown in FIG. 5, there was no statistically significant correlation between the plasma lipocalin-2 levels and the age of the control group (Spearman's correlation rho=0.042, p=0.807).

2-3. Comparison of Plasma Lipocalin-2 Levels and Cerebrospinal Fluid Lipocalin-2 Levels

Comparison was made between the lipocalin-2 levels in cerebrospinal fluid measured by the same method as the plasma lipocalin-2 levels to determine whether the change in the expression of lipocalin-2 levels in cerebrospinal fluid coincides with the change in the expression of lipocalin-2 levels in plasma.

The results are shown in the following table 3:

TABLE 3

| | Controls (n = 3) | MCI (n = 1) | AD (n = 4) |
|---|---|---|---|
| age range (mean) | 69-75 (72) | 78 | 56-66 (61) |
| LCN 2 range (mean, | 285-430 (342) | 853 | 193-673 (380) |
| pg/ml) | | | |

Abbreviation:
CSF, cerebrospinal fluid;
MCI, mild cognitive impairment;
AD, Alzheimer's disease.
Values are mean ± S.D

As shown in Table 3, the lipocalin-2 levels in cerebrospinal fluid were significantly increased in mild cognitive impairment patients compared to the Alzheimer's disease group and the control group.

Moreover, as shown in FIG. 6, although the lipocalin-2 level in cerebrospinal fluid was 100 or 1,000 fold higher compared to that in plasma, a similar pattern of changes in lipocalin-2 levels was found in the plasma and cerebrospinal fluid. In addition, there was a significant correlation between the plasma lipocalin-2 levels and cerebrospinal fluid lipocalin-2 levels.

Therefore, according to the present invention, it was found that the measurement of the level of lipocalin-2 expression in plasma and cerebrospinal fluid can be used as a biomarker for the diagnosis of mild cognitive impairment and can provide information for distinguishing mild cognitive impairment from Alzheimer's disease.

We claim:

1. A composition for diagnosing mild cognitive impairment, comprising an agent for measuring the level of mRNA or protein expression of lipocalin-2 gene.

2. The composition of claim 1, wherein the agent for measuring the level of protein expression comprises an antibody that is specific to lipocalin-2.

3. The composition of claim 1, wherein the agent for measuring the level of mRNA expression comprises a pair of primers or a probe that is specific to lipocalin-2 gene.

4. A kit for diagnosing mild cognitive impairment, comprising the composition of claim 1.

5. The kit of claim 4, wherein the kit is used to distinguish Alzheimer's disease from mild cognitive impairment.

6. A method for providing information for diagnosing mild cognitive impairment, characterized by measuring the level of lipocalin-2 protein expression.

7. The method of claim 6, wherein the method comprises the steps of:
measuring the level of lipocalin-2 protein in a biological sample; and
determining an increase in lipocalin-2 protein by comparison with a normal control group.

8. The method of claim 7, wherein the biological sample is plasma or cerebrospinal fluid.

9. The method of any one of claims 6 to 8, wherein the measurement is performed using an antibody against the lipocalin-2 protein.
